(19) United States
(12) Patent Application Publication
(10) Pub. No.: US 2013/0338776 A1
(43) Pub. Date: Dec. 19, 2013

(54) DEPLOYABLE INTERVERTEBRAL PROSTHESIS

(71) Applicant: David Bradley JONES, Redding, CA (US)

(72) Inventor: David Bradley JONES, Redding, CA (US)

(21) Appl. No.: 13/917,136

(22) Filed: Jun. 13, 2013

Related U.S. Application Data
(60) Provisional application No. 61/659,212, filed on Jun. 13, 2012.

(51) Int. Cl. A61F 2/44

(52) U.S. Cl. CPC A61F 2/442
USPC 623/17.16

(57) ABSTRACT

A device, system, and method for an expandable intervertebral prosthesis usable for spinal fixation. The prosthesis device generally includes at least two anchors that are a extendable from the device in opposing directions when a deployment pin is inserted into the device to displace the anchors. Implantation of the device may be facilitated by an implantation guide that is engageable with the device for device placement and accurate insertion of the pin into the device to extend the anchors.

### CROSS-REFERENCE TO RELATED APPLICATION

This application is related to and claims priority to U.S. Provisional Patent Application Ser. No. 61/659212, filed Jun. 13, 2012, entitled DEPLOYABLE INTERVERTEBRAL PROSTHESIS, the entirety of which is incorporated herein by reference.

### STATEMENT REGARDING FEDERALLY SPONSORED RESEARCH OR DEVELOPMENT

n/a

### FIELD OF THE INVENTION

The present invention relates to intervertebral spinal prostheses and methods of use and implantation thereof.

### BACKGROUND OF THE INVENTION

Spinal fusion is considered the “gold standard” for surgically treating patients whose condition has become so severe and debilitated that conservative, non-surgical measures fail to provide relief. Using bone grafts along with implants such as metal plates, rods and screws, spinal fusion adjoins two adjacent vertebrae, thus stabilizing the segment and easing the patient's pain, numbness, weakness and/or lack of mobility. Interbody fusion procedures in the cervical and lumbar spine can be approached from an anterior-based procedure, which includes removal of the intervertebral disc and appropriate preparation of the vertebral end plates for fusion. Traditionally, this type of surgery is referred to as an Anterior Lumbar Interbody Fusion (ALIF) and Anterior Cervical Discectomy and Fusion (ACDF). Typical implants employed for these procedures include a spacer made of either allograft or PEEK that resides in the removed disc space. Segmental fixation is then conducted to stabilize the vertebral segment through the use of either a separate anterior metal plate and screws for the cervical or lumbar spine, or placement of pedicle screws in the lumbar spine.

However, problems can arise with the anterior-placed hardware. Particularly, the implanted fixation hardware can cause post-operative morbidity issues with the overlying anatomy manifested as either swallowing discomfort in the cervical spine, issues with hardware impingement on adjacent level motion segments via subsidence of the hardware, and/or abundant scar tissue formation. The lumbar spine also poses potential risk with the great vessels and potential erosion into these vessels, as well as scarring that can also incorporate these vital anatomical structures.

In addition to the issues discussed above, implanting supplemental fixation hardware through the anterior approach can cause iatrogenic injury to the surrounding tissue, such as the major blood vessels in the neck and retroperitoneal abdomen as well as the esophagus. Many procedures often require additional retraction for proper screw alignment and placement through the plate fixation and into the respective vertebral body, which even further increases the likelihood that vital structures proximate the surgical site are injured.

Aside from hardware shortcomings and difficulties in the anterior approach itself, the time duration required to complete such procedures is also of concern. Not only is it desirable to reduce the amount of time it takes to treat a patient to reduce any unwanted risk associated with prolonged surgical procedures, but reducing treatment times also results in additional availability of surgical facilities and surgeons, leading to reduced costs for the healthcare system.

Accordingly, in light of the above, it is desirable to provide intervertebral spinal prostheses and methods of use and implantation thereof that avoid unwanted risks to surrounding anatomical structures, reduce prosthesis positioning difficulties or complexities, and minimize the procedure time to complete such an implantation.

### SUMMARY OF THE INVENTION

The present invention advantageously provides a device, system, and method for an intervertebral prosthesis that is expandable to engage adjacent vertebrae, such as may be used for spinal fixation. The intervertebral device may include a device body having a first surface and a second surface, and at least two anchors extendable from the first face and the second face in opposing directions. The device body may further have a first face and a second face, and the device may further include a conduit having an opening in the first face of the device body, the conduit extending within the device body between the first surface and the second surface, and at least a portion of each of the at least two anchors extending into the conduit. The anchors may be serrated on at least one surface, such as the surface that will be in contact with vertebrae adjacent to the implantation site. Further, the device body may define a recessed area in at least one of the first surface and the second surface. The first face may have a first width and the second face may have a second with, with the first width being greater than the second width. The device may further include a pin that is matable with the conduit, insertion of the pin into the conduit displacing the at least two anchors and extending the at least two anchors from the first face and the second face in opposing directions. For example, the pin and the conduit may be matably threaded and each of the at least two anchors may include at least one cam element, such that rotation of the pin within the conduit displaces the at least one cam element to extend the at least two anchors. The device may further include a face plate that is engageable with the first face of the device body, such that engagement of the face plate with the first face of the device body prevents the pin from exiting the conduit. Alternatively, the device may include a face plate that is integrated with the pin, such that insertion of the pin into the conduit brings the face plate in contact with the first face of the device body. The device may further include two pins and two conduits, and a radiopaque marker on the device body.

The system for spinal fixation may include a prosthesis device having: a device body having a first surface, a second surface, a first face, a second face, and at least two anchors slidably received within the device body; a first device conduit having an opening in the first face of the device body, the conduit extending within the device body between the first surface and the second surface, and at least a portion of each of the at least two anchors extending into the conduit; a second device conduit having an opening in the first face of the device body, the conduit extending within the device body between the first surface and the second surface; and a device shaft that is matable with the first device conduit. The system may further include an implantation guide having: a guide body having a first guide conduit and a second guide conduit, the device shaft being engageable with the first guide conduit and the first device conduit; and a guide shaft that is engageable the second guide conduit, the guide shaft further being engageable with the second device conduit, the first guide conduit and the first device conduit being substantially collinear when the guide and the device are in contact with each other, and the second guide conduit and the second device conduit being substantially collinear when the guide and the device are in contact with each other. The at least two anchors may be extendable from the first face and the second face of the device body. For example, insertion of the device shaft into the first device conduit may displace the at least two anchors and may extend the at least two anchors from the first face and the second face of the device body in opposing directions. In one embodiment, the guide body may be hollow and sized to receive the prosthesis device therein.

A method for implanting an intervertebral device may include passing a guide shaft through a first conduit of a guide frame through a first conduit of a prosthesis device to couple the guide frame to the prosthesis device, inserting the prosthesis device between two adjacent vertebrae, passing a device shaft through a second guide frame conduit and into a second prosthesis device conduit, insertion of the device shaft into the second prosthesis device conduit extending at least two anchors in opposing directions from the prosthesis device toward each of the two adjacent vertebrae, and removing the guide shaft from the first conduit of the prosthesis device to uncouple the guide frame and the prosthesis device. The prosthesis device may include a first face, and each of the first and second device conduits may have an opening in the first face of the prosthesis device. The method may further include coupling a face plate to the first face of the prosthesis device to cover the opening of the first device conduit and the opening of the second device conduit. The guide frame may include at least one stop element, and the method may further include inserting the prosthesis device between the two adjacent vertebrae until the at least one stop element is in contact with at least one of the two adjacent vertebrae. In one embodiment, the guide frame may be hollow and sized to receive the prosthesis device therein when the guide frame is coupled to the prosthesis device.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a top view (a view of the first surface) of an example of a spinal prosthesis constructed in accordance with present invention;

FIG. 2A is a front view of the first face of the spinal prosthesis of FIG. 1, with the anchors retracted;

FIG. 2B is a front view of the first face of the spinal prosthesis of FIG. 1, with the anchors extended;

FIG. 3 is a side view the spinal prosthesis of FIG. 1;

FIG. 4 is an examplary deployment pin constructed in accordance with present invention;

FIG. 5 is a first embodiment of an examplary prosthesis plate constructed in accordance with present invention;

FIG. 6 is a second embodiment of an exemplary prosthesis plate constructed in accordance with the present invention;

FIG. 7 is a top view of a first embodiment of an implant guide constructed in accordance with present invention;

FIG. 8 is a side view of the implant guide of FIG. 7 coupled to the spinal prosthesis of FIG. 1;

FIG. 9 is a top view of a second embodiment of an implant guide constructed in accordance with the present invention;

FIG. 10 is a side view of the implant guide of FIG. 9 coupled to the spinal prosthesis of FIG. 1;

FIG. 11 is a simplified side view of the implant within an intervertebral space, with the anchors retracted; and

FIG. 12 is a simplified side view of the implant within an intervertebral space, with the anchors extended.

### DETAILED DESCRIPTION OF THE INVENTION

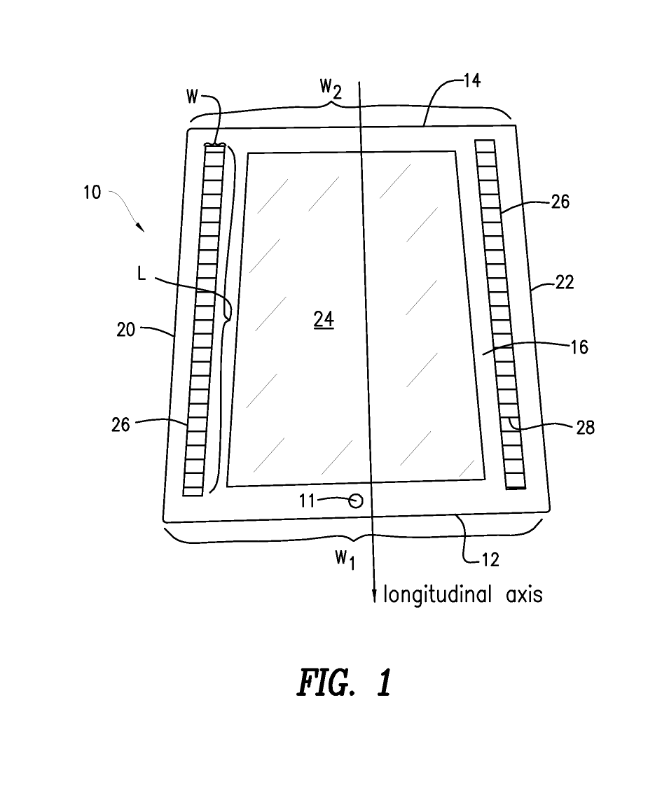

The present invention provides intervertebral spinal prostheses and methods of use and implantation thereof that avoid unwanted risks to surrounding anatomical structures, reduce prosthesis positioning difficulties or complexities, and minimize the procedure time to complete such an implantation. Referring now to the drawing figures, which may not be drawn to scale, in which like reference designations refer to like elements, a top view of an exemplary spinal prosthesis constructed in accordance with principles of the present invention is shown in FIG. 1, generally designated as “10.”

The prosthesis 10 may generally define a body implantable or otherwise positionable in an intervertebral space or location between two adjacent vertebrae. The spinal prosthesis 10 may be constructed from a range of biocompatible materials, including various polymers (such as polyether ether ketone, or PEEK), metals, and/or combinations thereof, and may include one or more radiopaque markers 11 or other features readily identifiable through medical imaging modalities. The prosthesis 10 may generally include a first face 12, a second face 14, a first surface 16, a second surface 18, a third surface 20, and a fourth surface 22. Dimensions of the prosthesis 10 may vary for particular applications and/or patients. For example, the prosthesis 10 may be available in a range of standard lordotic sizes for both cervical and lumbar procedures, and the appropriate prosthesis 10 may be selected depending on the patient's unique anatomy and/or the procedure to be performed.

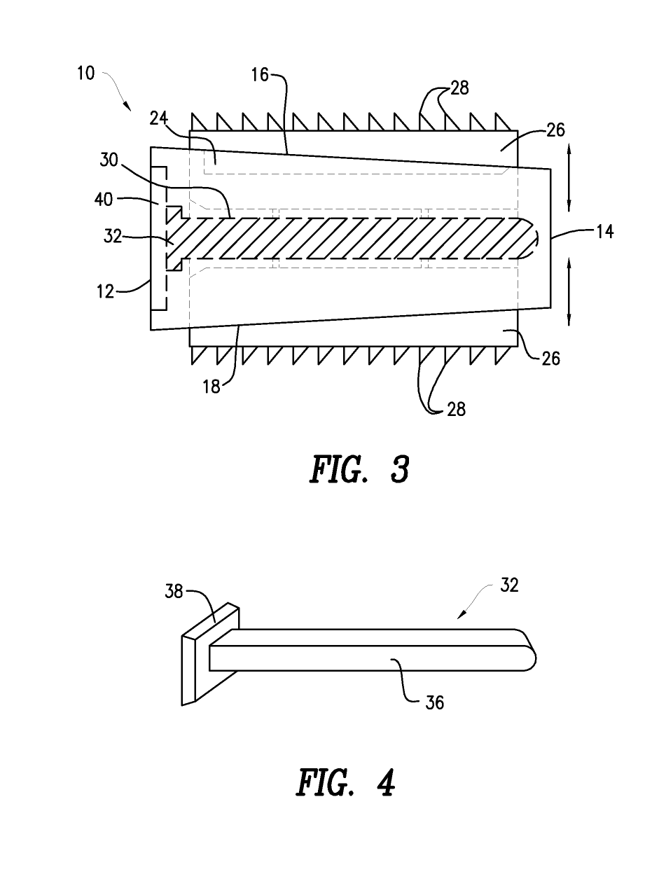

As is shown in FIG. 1, the first face 12 of the prosthesis 10 may have a width W₁ that is greater than the width W₂ of the second face 14 of the prosthesis 10, giving the prosthesis 10 a slanted or tapered cross-section along plane between the first and second faces 12, 14. The prosthesis may define a longitudinal axis between the first face 12 and the second face 14, and may also have a slanted or tapered cross-section along the longitudinal axis, as shown in FIG. 3, to facilitate positioning and implantation of the prosthesis 10 into a prepared surgical site, as well as to provide a selected degree of lordosis for the targeted spinal segment once implanted. Further, the prosthesis 10 may generally define a recessed area, opening, or channel 24 therethrough that allows for graft insertion and/or tissue in-growth about the prosthesis 10 when implanted.

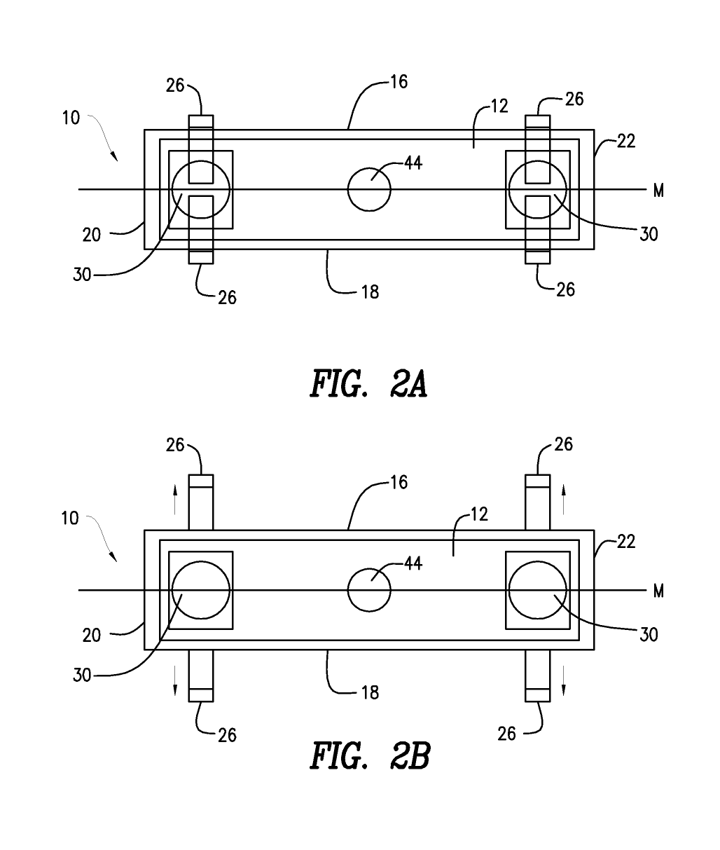

Referring now to FIGS. 1-2B, the prosthesis 10 may further include a selectively deployable anchoring mechanism facilitating secure positing and retention of the prosthesis 10 in a surgical site. For example, the prosthesis 10 may include at least two anchors 26 movably coupled to the body of the prosthesis 10. The anchors 26 may be selectively extendable in opposing directions away from the first surface 16 and the second surface 18 (as shown in FIG. 2) to engage tissue regions of the adjacent vertebrae. As used herein, “opposing directions” means that, in each pair of anchors 26 associated with the same passage 30, one anchor 26 will extend from the first surface 16 and the other anchor 26 will extend from the second surface 18 of the prosthesis 10. For example, the anchors 26 extend from the first surface 16 and second surface 18 in directions that are perpendicular (90°) or substantially perpendicular (for example, 90°±5°) from the surfaces 16, 18. Alternatively, the prosthesis 10 may be constructed such that the anchors 26 may be selectively extendable in opposing directions that are less than less than 90°, for example, at angles of between approximately 30° and up to 85°. Extending the anchors 26 at an angle may allow the anchors 26 to engage the adjacent vertebrae in a more stable manner, depending on the degree of lordosis and/or location of the prosthesis 10. Each anchor 26 may include a plurality of teeth or grooves 28 to provide a serrated surface and thereby enhance the tissue engagement and reduce the likelihood of movement or “backout” of the prosthesis 10 after implantation. Further, as a non-limiting example, each anchor 26 may define a length “L” and a width “W,” and the length L may be greater than the width W. When deployed, each anchor 26 may have the appearance of a plate that is perpendicular to the first 16 or second 18 surface of the prosthesis 10 (for example, as shown in FIG. 3).

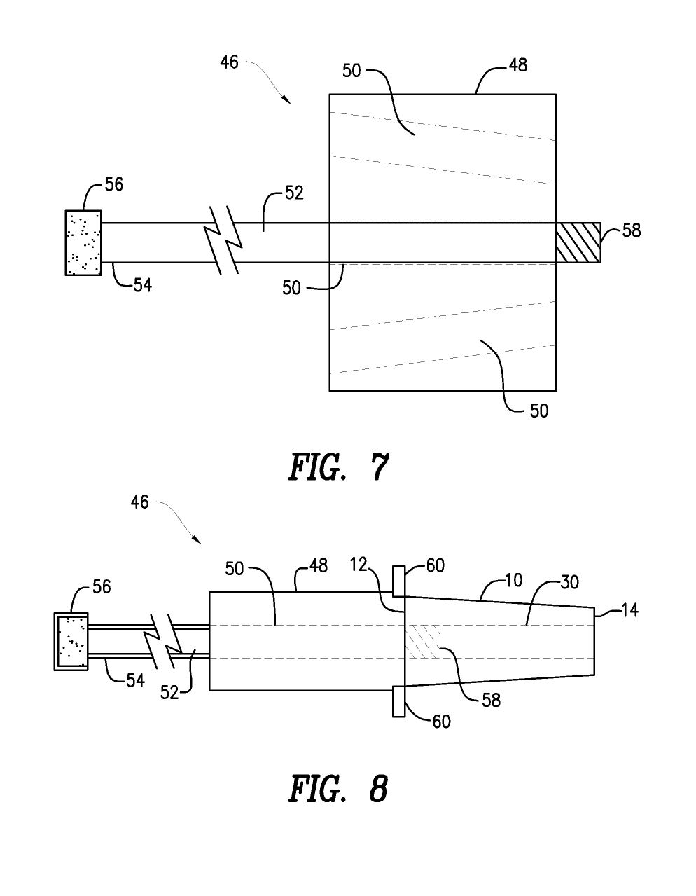

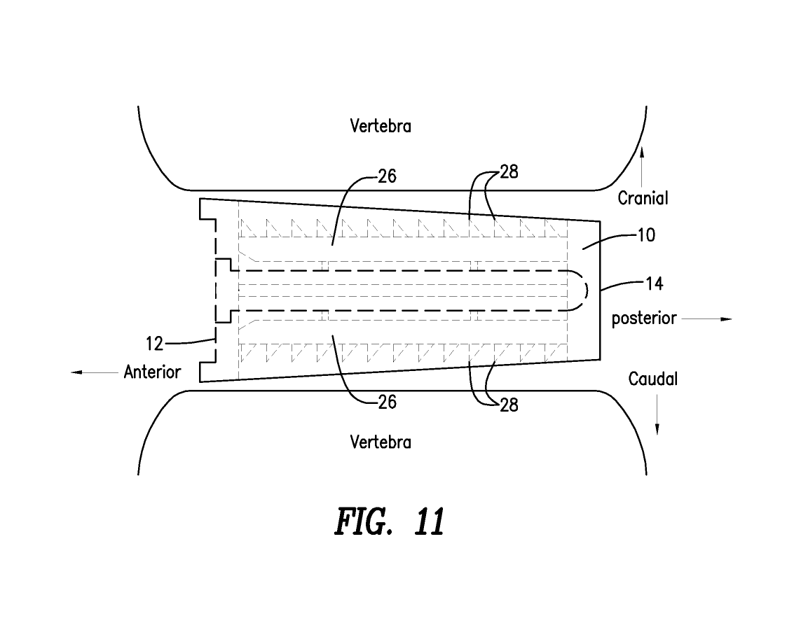

The selective deployment of the one or more anchors 26 may be achieved through manipulation or interaction with the first face 12 of the prosthesis 10, which avoids any need to expand the surgical site. For example, the prosthesis 10 may define one or more passages or conduits 30 extending through at least a portion of the prosthesis 10 in a substantially anterior-posterior direction (that is, substantially parallel to the longitudinal axis of the prosthesis 10). The one or more passages 30 may be positioned approximately midway between the first surface 16 and the second surface 18 of the prosthesis 10 (as shown in FIG. 2 as dashed line “M”). The one or more passages 30 may be substantially parallel to the longitudinal axis of the prosthesis 10 (for example, as shown in FIGS. 6 and 9) or the one or more passages 30 may be substantially parallel to the third surface 20 and the fourth surface 22 of the prosthesis 10 (as shown in FIG. 7), so that the passages 30 follow the tapered shape of the prosthesis 10 as shown in FIG. 1. Further, the one or more passages 30 may intersect or otherwise contain a portion of the one or more anchors 26 therein. For example, as shown in FIG. 2A, when the anchors 26 are in a retracted or partially retracted position (such as before a deployment pin 32 is inserted into the passage 30), a portion of each anchor 26 may lie within the passage 30. For clarity, FIG. 2A shows the anchors 26 as being partially retracted; however, when the anchors 26 are fully retracted, the serrations 28 of the anchors may lie entirely or substantially within the prosthesis 10 (as shown in FIG. 11). As shown in FIG. 2B, however, when the anchors 26 are in the extended position, the deployment pin 32 may displace the anchors 26 such that no portion or only a very small portion of each anchor 26 remains in the passage 30. The anchors 26 may be extendable in pairs, with a first anchor 26 extending from the first surface 16 of the prosthesis 10 and the second anchor 26 extending from the second surface 18 of the prosthesis when the deployment pin 32 is inserted into the passage 30, displacing both of the anchors 26 in opposite or substantially opposite directions.

Referring now to FIGS. 3 and 4, a side view of the spinal prosthesis and exemplary deployment pins are shown. One or more deployment pins 32 may be provided that are positionable within the passages 30 in order to deploy the anchors 26 (as shown in FIG. 3). Passages 30 may or may not extend all the way through the prosthesis 10 (for example, FIG. 3 shows the passage 30 extending partially through the prosthesis 10, from the first face 12 to the second face 14). A deployment pin 32 may be positioned within each of the passages 30 of the prosthesis 10, although fewer than all passages 30 may include a deployment pin 32. In particular, directing a deployment pin 32 into a passage 30 may displace an anchor 26, resulting in the cranial and/or caudal deployment or movement of the anchor 14 with respect to the first 16 or second 18 surface of the body of the prosthesis 10 (as indicated by the arrows shown in FIG. 3). In the non-limiting example in FIG. 3, the deployment pins 32 and the passages 30 may be threaded and anchors 26 may be deployable using a series of cams 34, such that rotation of the deployment pins 32 adjustable extends the anchors 26 from the first 16 and second 18 surfaces of the prosthesis 10. Alternatively, in the non-limiting example in FIG. 4, the deployment pins 32 may itself may physically displace the anchors 26. In that case, the anchors 26 may not include cams 34. Although the deployment pin 32 is shown in FIG. 4 has having a square or rectangular cross-section, it will be understood that the deployment pin 32 may have other cross-sectional shapes, such as circular or polygonal cross-sections. The deployment pin 32 of both FIGS. 3 and 4 may include a body 36 and a head 38 having a diameter that is wider than the body 36. Further, the body 36 of the deployment pin 32 may be pointed (as shown in FIG. 6) to separate the anchors 26, and/or the anchors 26 may include a beveled edge (as shown in FIG. 3) to facilitate entry of the deployment pin 32 into the passage 30 and separation of the anchors 26.

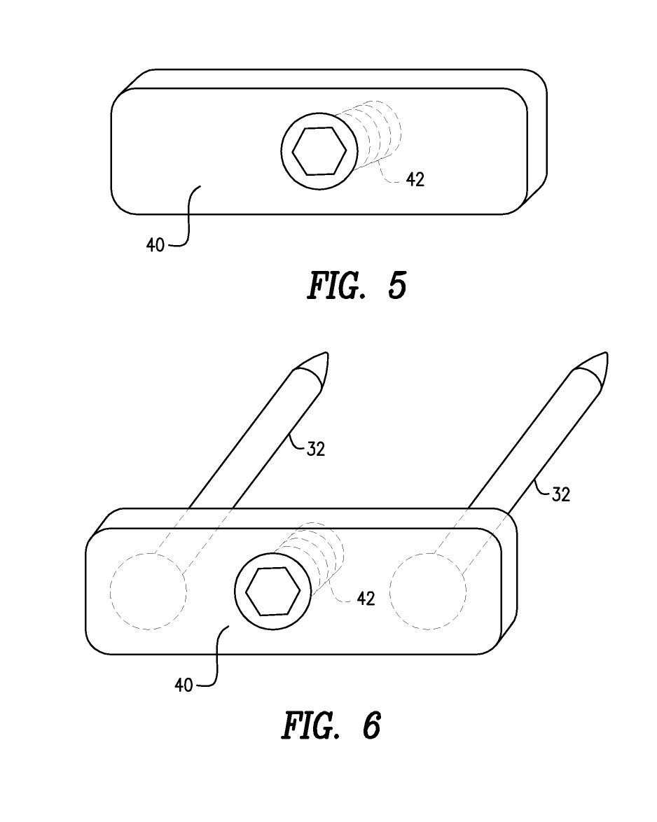

Referring now to FIGS. 5 and 6, a first and second embodiment of an exemplary prosthesis plate are shown. Once the deployment pins 32 are in place, a prosthesis plate 40 may be secured to the prosthesis 10 to lock the deployment pins 32 into position, preventing their movement or backing out after implantation. The prosthesis plate 40 may be releasably attachable to the first face 12 of the prosthesis 10, and may be secured to the body of the prosthesis 10 through a fastener 42 such as a screw, cam-lock mechanism, or the like, that mates with a complimentary receiving feature 44 of the prosthesis 10 (as shown in FIG. 2, for example). In an alternative configuration shown in FIG. 6, the prosthesis plate 40 and deployment pin(s) 32 may be integrated into a single or unitary construct that is inserted and fastened to the prosthesis 10 in a single step. In this embodiment, the one or more passages 30 of the prosthesis 10 may be substantially parallel to the longitudinal axis of the prosthesis 10.

Now referring to FIGS. 7-10, a top view and a side view of a first and second embodiment of an implant or prosthesis guide are shown. The prosthesis guide 46, 47 may be used to position the prosthesis 10 within a designate surgical site. For example, the prosthesis guide 46, 47 may be used to provide a perpendicular approach to the prosthesis 10 for the deployment pins 32 and prosthesis plate 40 with plate fastener 42. The guide 46, 47 may generally include a frame 48 engageable with the prosthesis 10, where the frame 48 defines one or more passages or conduits 50 alignable with the passages 30 of the prosthesis 10, thereby allowing one or more deployment pins 32 to be routed through the implant guide 46, 47 and into position within the prosthesis 10, as discussed in more detail below. The guide 46, 47 may further include a shaft 52 slidingly disposed within a passage 50 the frame 48 and releasably engageable with the prosthesis 10. The passage 50 within which the shaft 52 is disposed may be located at or near the middle of the frame 48, as shown in FIGS. 7-10. The shaft 52 may include a first end 54 defining or coupled to a handle 56, and a second end 58 that is threaded or contains other features for releasably mating with a portion of the prosthesis 10, such as the receiving feature 44. The guide 46, 47 may also include one or more “stops” 60 that reduce the likelihood of inserting the guide and/or prosthesis 10 farther into the surgical site than desired. For example, the stops 60 may extend from a portion of the frame 48 in a cranial-caudal direction to abut the upper and lower adjacent vertebrae defining the surgical site therebetween.

In the first prosthesis guide 46 embodiment shown in FIG. 7, the frame 48 may be solid and the passages 50 may extend entirely therethrough. When the guide 46 is engaged with the prosthesis 10 (as shown in FIG. 8), the frame 48 may abut the first face 12 of the prosthesis 10 and the passages 50 may be substantially collinear such that the deployment pins 32 may be smooth passed through the frame 48 and into the prosthesis 10, and the shaft 52 may be smooth passed through the frame 48 and engage with the prosthesis 10 (for example, the threaded second end 58 of the shaft 52 may be received by and engaged with a screw hole 44 in the first face 12 of the prosthesis 10).

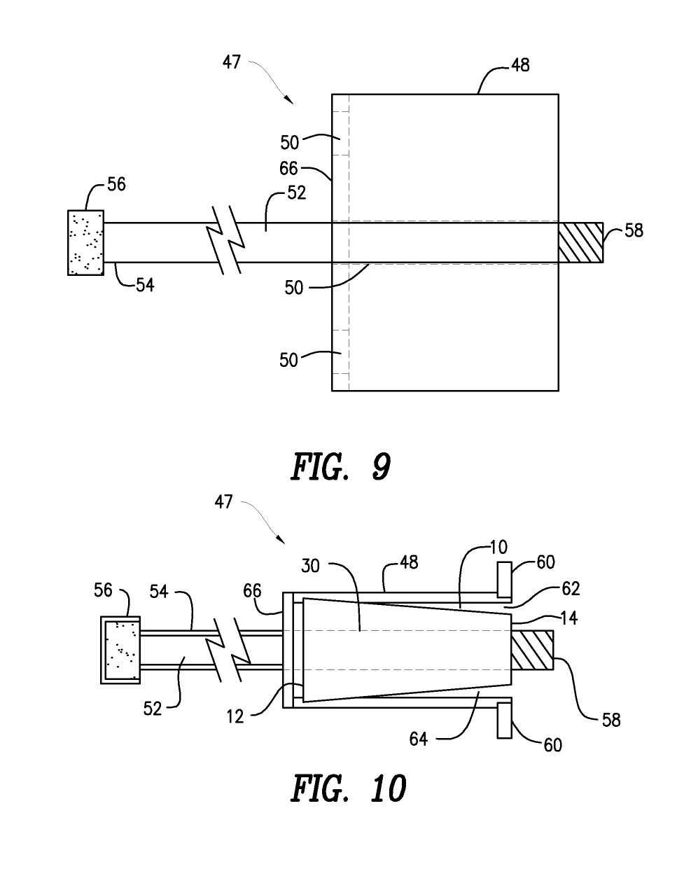

In the second prosthesis guide 47 shown in FIG. 9, the frame 48 may be hollow and include an open end 62 and open sides. The frame 48 may be sized to receive the prosthesis 10 within the frame 48 (as shown in FIG. 10). Further, the passages 50 may only extend through the closed end 66 of the frame 48, but, like the embodiment of FIGS. 7 and 8, the passages 50 may be collinear with the passages 30 of the prosthesis 10.

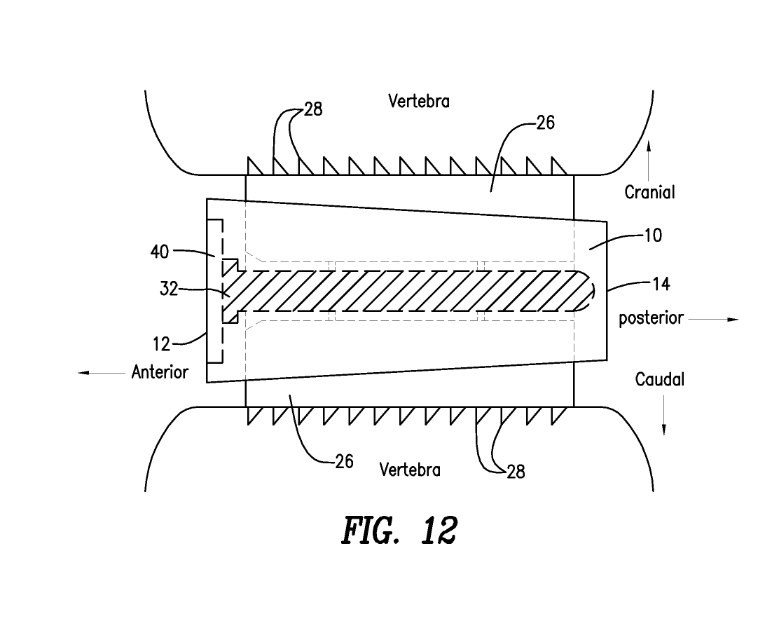

Referring now to FIGS. 11 and 12, the prosthesis 10 is shown within an intervertebral space. In an exemplary method of use, an intervertebral surgical site may be prepared, which may include resecting a portion of a diseased or damaged intervertebral disc. One or more slots or grooves may be cut into the adjacent vertebrae or remaining disc tissue in anticipation of receiving a portion of the prosthesis 10, such as the anchors 26. For example, the surgical site may be prepared such that it has a height that is slightly greater than that of the prosthesis 10 when the anchors 26 are retracted (as shown in FIGS. 2A and 11) and slightly less than that of the prosthesis 10, in particular, the anchors 26, when the anchors 26 are deployed (as shown in FIGS. 2B and 12). This allows the serrations 28 of the anchors 26 to engage with the tissue and/or bone of the adjacent vertebrae within the surgical site. The shaft 52 of the guide 46 may then be coupled to the prosthesis 10, and manipulation of the handle 56 can allow for insertion and precise positioning of the prosthesis 10 into the prepared surgical space through an anterior approach. As shown in FIGS. 11 and 12, the prosthesis 10 may be inserted in an anterior-to-posterior direction. However, other approaches may also be used. Prior to insertion, the opening or channel 24 within the prosthesis 10 may be filled with graft material (which may include autograft, allograft, xenograft, and/or isograft material). During positioning and insertion, the anchors 26 may be in a retracted state, which minimizes the insertion profile of the prosthesis 10 and eases the overall entry and positioning of the prosthesis 10. The stops 60 of the guide 46, 47 may ensure that the prosthesis 10 is not inserted too far into the surgical space, and fluoroscopy or other imaging means may be used to exactly position the prosthesis 10. Once the desired positioning is achieved, one or more deployment pins 32 may be passed through the frame 48 of the prosthesis guide 46, 47 and into the respective passages 30 of the prosthesis 10, thereby deploying the anchors 26 outward to securely engage the surrounding tissue. The length of the deployment pins 32 may be such that when the deployment pins 32 are positioned within the prosthesis 10, no part of the deployment pins 32 remains within the guide 46, 47. The shaft 52 may be decoupled from the prosthesis 10 and the prosthesis plate 40 may then be secured to the first face 12 of the prosthesis 10 to lock the deployment pins 32, and thus the anchors 26, in place. Alternatively, if the integrated prosthesis plate of FIG. 6 is used, the prosthesis guide 46 may first be removed and then the prosthesis plate 40 with integrated deployment pins 32 may be coupled to the prosthesis 10 in a single step.

The present invention provides a device that includes both the spacing and fixation hardware and is substantially or entirely contained within the intervertebral disc space. Thus, the device does not interfere with spinal anatomy surrounding the implantation location. Further, the procedures for preparing the disc and vertebral endplates are not significantly altered from a spinal surgeon's standard technique. That is, the standard approach for an ALIF or ACDF procedure may be used. Finally, the present invention reduces the morbidity risk to surrounding neurovascular tissue and other soft tissues, thus allowing for faster procedure and patient recovery times while still achieving a stable and secure fixation for fusion.

It will be appreciated by persons skilled in the art that the present invention is not limited to what has been particularly shown and described herein above. In addition, unless mention was made above to the contrary, it should be noted that all of the accompanying drawings are not to scale. Of note, the system components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein. Moreover, while certain embodiments or figures described herein may illustrate features not expressly indicated on other figures or embodiments, it is understood that the features and components of the system and devices disclosed herein are not necessarily exclusive of each other and may be included in a variety of different combinations or configurations without departing from the scope and spirit of the invention. A variety of modifications and variations are possible in light of the above teachings without departing from the scope and spirit of the invention, which is limited only by the following claims.

What is claimed is:

1. An intervertebral device, the device comprising:
a device body having a first surface and a second surface; and
at least two anchors extendable from the first face and the second face in opposing directions.

2. The device of claim 1, wherein the device body further has a first face and a second face, the device further comprising:
a conduit having an opening in the first face of the device body, the conduit extending within the device body between the first surface and the second surface, and at least a portion of each of the at least two anchors extending into the conduit.

3. The device of claim 2, wherein the anchors are serrated on at least one surface.

4. The device of claim 2, wherein the device body defines a recessed area in at least one of the first surface and the second surface.

5. The device of claim 2, wherein the first face has a first width and the second face has a second width, the first width being greater than the second width.

6. The device of claim 2, further comprising a pin that is matable with the conduit, insertion of the pin into the conduit displacing the at least two anchors and extending the at least two anchors from the first face and the second face in opposing directions.

7. The device of claim 6, wherein the pin and the conduit are matably threaded and each of the at least two anchors include at least one cam element, such that rotation of the pin within the conduit displaces the at least one cam element to extend the at least two anchors.

8. The device of claim 6, further comprising a face plate that is engageable with the first face of the device body, such that engagement of the face plate with the first face of the device body prevents the pin from exiting the conduit.

9. The device of claim 6, further comprising a face plate that is integrated with the pin, such that insertion of the pin into the conduit brings the face plate in contact with the first face of the device body.

10. The device of claim 6, wherein the device comprises two pins and two conduits.

11. The device of claim 1, further comprising a radiopaque marker on the device body.

12. A system for spinal fixation, the system comprising:
a prosthesis device including:
a device body having a first surface, a second surface, a first face, a second face, and at least two anchors slidably received within the device body;
a first device conduit having an opening in the first face of the device body, the conduit extending within the device body between the first surface and the second surface, and at least a portion of each of the at least two anchors extending into the conduit;
a second device conduit having an opening in the first face of the device body, the conduit extending within the device body between the first surface and the second surface; and
a device shaft that is matable with the first device conduit; and
an implantation guide including:
a guide body having a first guide conduit and a second guide conduit, the device shaft being engageable with the first guide conduit and the first device conduit; and
a guide shaft that is engageable the second guide conduit, the guide shaft further being engageable with the second device conduit,
the first guide conduit and the first device conduit being substantially collinear when the guide and the device are in contact with each other, and the second guide conduit and the second device conduit being substantially collinear when the guide and the device are in contact with each other.

13. The system of claim 12, wherein the guide body is hollow and sized to receive the prosthesis device therein.

14. The system of claim 12, wherein the at least two anchors are each serrated on at least one surface.

15. The system of claim 12, wherein the at least two anchors are extendable from the first face and the second face of the device body.

16. The system of claim 15, wherein insertion of the device shaft into the first device conduit displaces the at least two anchors and extends the at least two anchors from the first face and the second face of the device body in opposing directions.

17. A method for implanting an intervertebral device, the method comprising:
passing a guide shaft through a first conduit of a guide frame through a first conduit of a prosthesis device to couple the guide frame to the prosthesis device;
inserting the prosthesis device between two adjacent vertebrae;
passing a device shaft through a second guide frame conduit and into a second prosthesis device conduit, insertion of the device shaft into the second prosthesis device conduit extending at least two anchors in opposing directions from the prosthesis device toward each of the two adjacent vertebrae; and
removing the guide shaft from the first conduit of the prosthesis device to uncouple the guide frame and the prosthesis device.

18. The method of claim 17, wherein the prosthesis device includes a first face, and each of the first and second device conduits have an opening in the first face of the prosthesis device, the method further comprising coupling a face plate to the first face of the prosthesis device to cover the opening of the first device conduit and the opening of the second device conduit.

19. The method of claim 17, wherein the guide frame is hollow and sized to receive the prosthesis device therein when the guide frame is coupled to the prosthesis device.

20. The method of claim 17, wherein the guide frame includes at least one stop element, the method further comprising inserting the prosthesis device between the two adjacent vertebrae until the at least one stop element is in contact with at least one of the two adjacent vertebrae.
